(19) United States
(12) Patent Application Publication
(10) Pub. No.: US 2006/0111286 A1
(43) Pub. Date: May 25, 2006

(54) Method of modulating endothelial cell activity

(76) Inventors: Jennifer Ruth Gamble, Stirling (AU); Mathew Alexander Vadas, Stirling (AU); Li Ziaochun, Adelaide (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/526,812

(22) PCT Filed: Sep. 5, 2003
(86) PCT No.: PCT/AU03/01154

§ 371 Date: Aug. 19, 2005

(30) Foreign Application Priority Data
Sep. 6, 2002 (AU) 2002951253

(51) Int. Cl. A61K 38/18; A61K 31/407

(52) U.S. Cl. 514/12; 514/410

(57) ABSTRACT

The present invention relates generally to a method of modulating endothelial cell activity and to agents useful for same. More particularly, the present invention relates to a method of modulating intercellular vascular endothelial permeability by modulating an intracellular protein kinase C-dependent signalling mechanism. The method for the present invention is useful, inter alia, in the treatment and/or prophylaxis of a condition charicterised by aberrant, unwanted or otherwise inappropriate endothelial cell activity, in particular, conditions characterised by a loss of vascular intergrity.

### FIELD OF THE INVENTION

The present invention relates generally to a method of modulating endothelial cell activity and to agents useful for same. More particularly, the present invention relates to a method of modulating intercellular vascular endothelial permeability by modulating an intracellular protein kinase C-dependent signalling mechanism. The method for the present invention is useful, inter alia, in the treatment and/or prophylaxis of a condition characterised by aberrant, unwanted or otherwise inappropriate endothelial cell activity, in particular, conditions characterised by a loss of vascular integrity.

### BACKGROUND OF THE INVENTION

Bibliographic details of the publications referred to by author in this specification are collected alphabetically at the end of the description.

The reference to any prior art in this specification is not, and should not be taken as, an acknowledgment or any form of suggestion that that prior art forms part of the common general knowledge in Australia or any other country.

One of the essential functions of the endothelial cell lining is to maintain the essentially impermeable nature of the blood vessel controlling the passage of solutes and inflammatory cells from the circulation to the tissues. Endothelial cell hyper-permeability is a characteristic of blood vessels in many pathologies. For example, newly formed micro-vessels in tumours are highly permeable. Indeed, such hyper-permeability allows the deposition of fibrin in tumours that supports and promotes cell adhesion and migration, essential steps in the angiogenic response (Dvorak, H. F., Harvey, V. S., Estrella, P., Brown, L. F., McDonagh, J., Dvorak, A. M. (1987) Lab Invest. 57:673-86; Dvorak, H. F., Brown, L. F., Detmar, M., Dvorak, A. M. (1995) Am J Pathol. 146:1029-39). In chronic inflammatory states such as in rheumatoid arthritis and atherosclerosis, vessel hyper-permeability allows increased transmigration of inflammatory cells across the activated endothelium. A number of factors have previously been described which promote endothelial cell leakiness, for example, thrombin, tumour necrosis-factor and vascular endothelial cell growth factor (VEGF). These appear to act by inducing changes in junctional molecules such as PECAM-1 and VE-cadherin or their associated signalling molecules, such as the catenins.

Thrombin is a serine protease with multiple roles central to vascular biology, acting upon platelets, endothelial cells and circulating clotting factors (Macfarlane, S. R., Seatter, M. J., Kantce, T., Hunter, G. D., Plevin, R. (2001) Pharmacol Rev. 53:245-82). Thrombin is a potent activator of endothelial cells, increasing intercellular gap formation and the permeability of confluent endothelial cell monolayers (Lum, H., Andersen, T. T., Siflinger-Birnboim, A., Tiruppathi, C., Goligorsky, M. S., Fenton, J. W. 2nd, Malik, A. B. (1993) J Cell Biol. 120:1491-9; Garcia, J. G., Verin, A. D., Schaphorst, K. L. (1996) Semin Thromb Hemost. 22:309-15). Thrombin signaling is mediated by the protease-activated receptor PAR-1 (Coughlin et al. (2000) supra). Thrombin cleaves the PAR-1 ligand from the receptor thus allowing the ligand to activate receptor signaling. PAR-1 can activate a number of downstream signaling pathways, the molecules activated depend upon the G-proteins recruited to the receptor (Maefarlane et al. (2001) supra).

The protein kinase Cζ family of serine/threonine kinases are involved in signal transduction and are dependent upon lipids for their activity. The isoforms of protein kinase Cζ are classified according to their structure and activation/substrate requirements (Draijer, R., Atsma, D. E., van der Laarse, A., van Hinsbergh, V. W. (1995) Circ Res. 76:199-208). The classical protein kinase Cζs (α, βI, βII, γ) are Ca²⁺-dependent and regulated by diacyglycerol (DAG) or phosphotidylserine (PS), the novel protein kinase Cζs (δ, ε, η, θ) are also activated by DAG or PS but are Ca²⁺-independent, while the atypical protein kinase Cζs (ζ, 1/λ) are regulated by PS, independent of both DAG and Ca²⁺. ¹⁰ The activity of protein kinase Cζs is controlled by their phosphorylation status and relocalisation (Parelk, D. B., Ziegler, W., Parker, P. J. (2000) Embo J. 19:496-503). The biological consequences of activation of particular isoforms of protein kinase Cζ under specific conditions have not yet been determined.

Protein kinase C has been demonstrated to function as one such PAR-1 downstream intermediate (Malik, 1994). However, studies of the effects of protein kinase C activation on endothelial cell permeability have produced conflicting results with some authors concluding that protein kinase C activation does not play a significant role in permeability changes in endothelial cells (van Nieuw Amerongen G P, Draijer R, Vermeer M A, van Hinsbergh V W. (1998) Circ Res. 83:1115-23; Vouret-Craviari V, Boquet P, Pouyssegur J, Van Obberghen-Schilling E. (1998) Mol Biol Cell. 9:2639-53) while others conclude that protein kinase C activation is important (Lynch J J, Ferro T J, Blumenstock F A, Brockenauer A M, Malik A B. (1990) J Clin Invest. 85:1991-8; Hempel, 1997). Studies using phorbol-12-myristate-13-acetate as a diacylglycerol surrogate to stimulate protein kinase C activation have shown both inhibition (Yamada Y, Furumichi T, Furui H, Yokoi T, Ito T, Yamauchi K, Yokota M, Hayashi H, Saito H. (1990) Arteriosclerosis 10:410-20; van Nieuw Amerongen, 1998 supra) and stimulation (Lynch, 1990 supra; Bussolino F, Silvagno F, Garbarino G, Costamagna C, Sanavio F, Arese M, Soldi R, Aglietta M, Pescannona G, Camussi G, et al. (1994) J Biol Chem. 269:2877-86; van Nieuw Amerongen, 1998 supra) of endothelial cell permeability in a concentration dependent manner (Lynch, 1990 supra; van Nieuw Amerongen, 1998 supra). However, phorbol esters do not activate atypical protein kinase Cs (Zhou G, Wooten M W, Coleman E S. (1994) Exp Cell Res. 214:1-11).

The signals which regulate endothelial cell permeability have clearly not been fully defined. However, elucidation of these cellular signalling mechanisms is essential for the development of therapeutic and/or prophylactic strategies directed to treating conditions characterised by aberrant or otherwise unwanted endothelial cell permeability.

In work leading up to the present invention, it has been determined that the atypical protein kinase Cζ plays a functional role in the regulation of endothelial cell permeability. In particular, it has been surprisingly determined that signalling relating to modulation of intercellular endothelial cell permeability critically involves activation of this atypical form of protein kinase C, resulting inter alia, in increased intercellular endothelial cell permeability. Since endothelial cell integrity is influenced by a balance between the influence of inflammatory mediators (such as thrombin) which increase intercellular gap formation and promotes endothelial permeability and anti-inflammatory agents which promote cell cell junction formation and antagonise changes in endothelial cell permeability, the elucidation of this cellular signalling mechanism now facilitates the rational design of methodology directed to modulating endothelial cell activity by regulating the functioning of protein kinase Cζ.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein, the term “derived from” shall be taken to indicate that a specified integer may be obtained from a particular source albeit not necessarily directly from that source.

One aspect of the present invention is directed to a method of modulating endothelial cell activity, said method comprising modulating the functional activity of protein kinase Cζ wherein up-regulating protein kinase Cζ activity to a functionally effective level up-regulates said cellular activity and down-regulation protein kinase Cζ activity to a functionally ineffective level down-regulates said cellular activity.

There is more particularly provided a method of modulating vascular endothelial cell activity, said method comprising modulating the functional activity of protein kinase Cζ wherein up-regulating protein kinase Cζ activity to a functionally effective level up-regulates said vascular endothelial activity and down-regulating protein kinase Cζ activity to a functionally ineffective level down-regulates said vascular endothelial cell activity.

In another aspect there is provided a method of modulating intercellular vascular endothelial cell permeability, said method comprising modulating the functional activity of protein kinase Cζ wherein up-regulating protein kinase Cζ to a functionally effective level up-regulates said intercellular vascular cell permeability and down-regulating protein kinase Cζ activity to a functionally ineffective level down-regulates said intercellular vascular endothelial cell permeability.

Yet another aspect provides a method of modulating thrombin-induced vascular endothelial cell activity, said method comprising modulating the functional activity of protein kinase Cζ wherein up-regulating protein kinase Cζ activity to a functionally effective level up-regulates said vascular endothelial activity and down-regulating protein kinase Cζ activity to a functionally ineffective level down-regulates said vascular endothelial cell activity.

Still another aspect provides a method of modulating thrombin induced intercellular vascular endothelial cell permeability, said method comprising modulating the functional activity of protein kinase Cζ wherein up-regulating protein kinase Cζ activity to a functionally effective level up-regulates said permeability and down-regulating said protein kinase Cζ activity to a functionally ineffective level down-regulates said permeability.

Yet still another aspect of the present invention is directed to a method of regulating endothelial cell activity in a mammal, said method comprising modulating the functional activity of protein kinase Cζ in said mammal wherein up-regulating protein kinase Cζ activity to a functionally effective level up-regulates said endothelial cell activity and down-regulating protein kinase Cζ activity to a functionally ineffective level down-regulates said endothelial cell activity.

In still yet another aspect there is provided a method of modulating vascular endothelial cell activity in a mammal, said method comprising modulating the functional activity of protein kinase Cζ wherein up-regulating protein kinase Cζ activity to a functionally effective level up-regulates said endothelial cell activity and down-regulating protein kinase Cζ activity to a functionally ineffective level down-regulates said endothelial cell activity.

In a further aspect there is provided a method of up-regulating vascular endothelial cell activity in a mammal, said method comprising administering to said mammal an effective amount of an agent for a time and under conditions sufficient to induce a functionally effective level of protein kinase Cζ.

In another further aspect there is provided a method of up-regulating vascular endothelial cell activity in a mammal, said method comprising administering to said mammal an effective amount of protein kinase Cζ for a time and under conditions sufficient to induce a functionally effective level of protein kinase Cζ.

In still another further aspect there is provided a method of up-regulating vascular endothelial cell activity in a mammal, said method comprising administering to said mammal an effective amount of a nucleotide sequence encoding protein kinase Cζ for a time and under conditions sufficient to induce a functionally effective level of protein kinase Cζ.

In yet still another aspect there is provided a method of down-regulating vascular endothelial cell activity in a mammal, said method comprising administering to said mammal an effective amount of an agent for a time and under conditions sufficient to induce a functionally ineffective level of protein kinase Cζ.

Another aspect of the present invention contemplates a method for the treatment and/or prophylaxis of a condition characterised by aberrant, unwanted or otherwise inappropriate endothelial cell activity in a mammal, said method comprising modulating the functional activity of protein kinase Cζ wherein up-regulating protein kinase Cζ activity to a functionally effective level up-regulates said endothelial cell activity and down-regulating protein kinase Cζ activity to a functionally ineffective level down-regulates said cellular activity.

In yet another aspect there is provided a method for the treatment and/or prophylaxis of a condition characterised by unwanted vascular endothelial cell activity in a mammal, said method comprising administering to said mammal an effective amount of an agent for a time and under conditions sufficient to induce a functionally ineffective level of protein kinase Cζ.

Still another aspect of the present invention relates to the use of an agent capable of modulating the functionally effective level of protein kinase Cζ in the manufacture of a medicament for the regulation of thrombin-induced endothelial cell activity in a mammal wherein up-regulating protein kinase Cζ activity to a functionally effective level up-regulates said endothelial cell activity and down-regulating protein kinase Cζ activity to a functionally ineffective level down-regulates said endothelial cell activity.

In another aspect the present invention relates to the use of protein kinase Cζ or a nucleic acid encoding protein kinase Cζ in the manufacture of a medicament for the regulation of endothelial cell activity wherein up-regulating protein kinase Cζ to a functionally level up-regulates said endothelial cell activity.

In yet another further aspect, the present invention contemplates a pharmaceutical composition comprising the modulatory agent as hereinbefore defined and one or more pharmaceutically acceptable carriers and/or diluents. Said agents are referred to as the active ingredients

### BRIEF DESCRIPTION OF THE DRAWINGS

FIG. 1 is a graphical representation of protein kinase C inhibitors selectively blocking thrombin stimulated permeability increases. Cells were untreated (Nil), treated with thrombin (0.2 U/ml), (T) for 15 mins, pre-treated with inhibitor for 15 mins or pre-treated with inhibitor followed by thrombin. a, Bisindolylmaleimide I blocks thrombin-stimulated permeability increases in endothelial cells at high concentrations (BisLo, 100 nM; BisHi, 6 μM). b, Chelerythrine chloride blocks thrombin-stimulated permeability increases in endothelial cells (Cζ, 1 μm). c, The specific PKA inhibitor H-89 has no effect on thrombin-stimulated permeability increases in endothelial cells (H-89, 50 nM).d, Calphostin C does not block thrombin-stimulated permeability increases in endothelial cells (CalC, 100 nM). Relative fluorescent emission at 530 nm is shown. Data are mean ±SEM of duplicate determinations in each group and are representative of at least 3 independent experiments.

FIG. 2 is an image of the treatment of endothelial cells with thrombin inducing protein kinase Cζ relocalisation. Endothelial cell monolayers were stained to demonstrate changes in protein kinase Cζ (a-c)and protein kinase Cζ (d-f) localization following treatment with thrombin (0.2 U/ml). a, control antibody. b, untreated. c, post thrombin treatment. d, control antibody. e, untreated. f, post thrombin treatment.

FIG. 3a is a graphical representation of dominant-negative protein kinase Cζ blocks thrombin stimulated permeability increases in endothelial cells. Endothelial cell were infected with pAdEasy-1 adenovirus empty vector (EV) or pAdEasy-1 constructs encoding dominant-negative protein kinase Cζ (DN) or constitutively active protein kinase Cζ (CA) prior to assay. Cells were either untreated (−) or treated with thrombin at 0.2 U/ml (+). Relative fluorescent emission at 530 nm is shown. Data shown are mean ±SEM of triplicate determinations and are representative of 3 independent experiments.

FIG. 3b is a graphical representation of angiopoietin-1 inhibiting thrombin stimulated endothelial cell permeability increases. Cells were untreated (Nil), treated with angiopoietin-1 (0.1 μl/ml) (Ang-1) for 30 min, treated with thrombin (0.2 U/ml), (Thrombin) for 15 mins, pre-treated with angiopoietin-1 (0.1 μg/ml) for 30 min followed by thrombin (Ang-1+T). Relative fluorescent emission at 530 nm is shown. Data shown are mean ±SEM of triplicate determinations and are representative of 3 independent experiments.

FIG. 4 is an image of angiopoietin-1 blocking thrombin stimulated protein kinase Cζ relocalisation. Endothelial cell monolayers were stained to demonstrate changes in protein kinase Cζ localisation following treatment with thrombin (0.2 U/ml) either alone or after pre-treatment with angiopoietin-1 (0.1 μg/ml), the pan-protein kinase C inhibitor bisindolylmaleimide I (6 μM) or the PI3-kinase inhibitor LY294002 (10 μM). a, No treatment. b, Thrombin alone. c, angiopoietin-1 and thrombin. d, bisindolylmaleimide I and thrombin. e, LY294002 and thrombin.

FIG. 5 is an image of angiopoietin-1 blocking thrombin stimulated protein kinase Cζ phoshorylation. a) Endothelial cells were infected with FLAG-protein kinase Cζ in the pAdEasy-1 adenovirus vector. Cells were lysed after treatment with thrombin (0.2 U/ml) either alone or after pre-treatment with angiopoietin-1 (0.1 μg/ml), the pan-protein kinase C inhibitor bisindolylmaleimide 1 (6 μM) or the PI3-kinase inhibitor LY294002 (10 μM). Proteins were separated by SDS-PAGE before transfer to PVDF membrane and probed with an antibody directed against phosphorylated Thr410 of the activation loop of protein kinase Cζ (upper panel). Membranes were stripped and re-probed with an anti-protein kinase Cζ antibody (lower panel). Nil, No treatment. T, Thrombin alone. Ang-1+T, angiopoietin-1 and thrombin. Bis+T, bisindolylmaleimide I and thrombin. LY+T, LY294002 and thrombin. b) Endothelial cells infected with pAdEasy-1 constructs encoding dominant-negative protein kinase Cζ or constitutively active protein kinase Cζ. Cells were either untreated or treated with thrombin, lysed and processed as for 5a. DN, dominant-negative protein kinase Cζ over-expressing cells. DN+T, dominant-negative protein kinase Cζ over-expressing cells and thrombin treated. CA, constitutively active protein kinase Cζ over-expressing cells. CA+T, constitutively active protein kinase Cζ over-expressing cells and thrombin treated.

FIG. 6 is a graphical representation of the PI3-kinase inhibitor LY294002 blocking thrombin-stimulated permeability increases in endothelial cells. Cells were untreated (Nil), treated with thrombin (0.2 U/ml), (T) for 15 mins, pre-treated with LY294002 (10 μM) (LY) for 15 mins or pre-treated with LY294002 followed by thrombin (LY+T). (+). Relative fluorescent emission at 530 nm is shown. Data shown are mean ±SEM of triplicate determinations and are representative of 3 independent experiments.

### DETAILED DESCRIPTION OF THE INVENTION

The present invention is predicated, in part, on the determination that the endothelial cell signalling related to modulation of intercellular permeability critically involves activation of the atypical protein kinase Cζ. This development now permits the rational design of therapeutic and/or prophylactic methods for treating conditions characterised by aberrant or unwanted endothelial cell activity.

Accordingly, one aspect of the present invention is directed to a method of modulating endothelial cell activity, said method comprising modulating the functional activity of protein kinase Cζ wherein up-regulating protein kinase Cζ activity to a functionally effective level up-regulates said cellular activity and down-regulation protein kinase Cζ activity to a functionally ineffective level down-regulates said cellular activity.

Reference to “endothelial cell” should be understood as a reference to the cells which line the blood vessels, lymphatics or other serous cavities such as fluid filled cavities. The phrase “endothelial cells” should also be understood as a reference to cells which exhibit one or more of the morphology, phenotype and/or functional activity of endothelial cells and is also a reference to mutants or variants thereof. “Variants” include, but are not limited to, cells exhibiting some but not all of the morphological or phenotypic features or functional activities of endothelial cells at any differentiative stage of development. “Mutants” include, but are not limited to, endothelial cells which have been naturally or non-naturally modified such as cells which are genetically modified.

It should also be understood that the endothelial cells of the present invention may be at any differentiative stage of development. Accordingly, the cells may be immature and therefore functionally incompetent in the absence of further differentiation. In this regard, highly immature cells such as stem cells, which retain the capacity to differentiate into endothelial cells, should nevertheless be understood to satisfy the definition of “endothelial cell” as utilised herein due to their capacity to differentiate into endothelial cells under appropriate conditions. Preferably, the subject endothelial cell is a vascular endothelial cell or a lymphatic endothelial cell.

Accordingly, there is more particularly provided a method of modulating vascular endothelial cell activity, said method comprising modulating the functional activity of protein kinase Cζ wherein up-regulating protein kinase Cζ activity to a functionally effective level up-regulates said vascular endothelial activity and down-regulating protein kinase Cζ activity to a functionally ineffective level down-regulates said vascular endothelial cell activity.

There is also provided a method of modulating lymphatic endothelial cell activity, said method comprising modulating the functional activity of protein kinase Cζ wherein up-regulating protein kinase Cζ activity to a functionally effective level up-regulates said lymphatic endothelial activity and down-regulating protein kinase Cζ activity to a functionally ineffective level down-regulates said lymphatic endothelial cell activity.

Reference to endothelial cell “activity” should be understood as a reference to any one or more of the functional activities which an endothelial cell is capable of performing, for example, as a result of stimulation by an extracellular agent such as thrombin, VEGF or TNF. Preferably, said activity is endothelial cell permeability.

Without limiting the present invention to any one theory or mode of action, it is thought that modulation of endothelial cell permeability can occur at the level of modulating intercellular permeability and/or intracellular permeability. It should be understood that in a preferred embodiment, the present invention is directed to modulation of one or both of these mechanisms of permeability.

According to this preferred embodiment there is provided a method of modulating intercellular endothelial cell permeability, said method comprising modulating the functional activity of protein kinase Cζ wherein up-regulating protein kinase Cζ to a functionally effective level up-regulates said intercellular cell permeability and down-regulating protein kinase Cζ activity to a functionally ineffective level down-regulates said intercellular endothelial cell permeability.

In another preferred embodiment there is provided a method of modulating intracellular endothelial cell permeability, said method comprising modulating the functional activity of protein kinase Cζ wherein up-regulating protein kinase Cζ to a functionally effective level up-regulates said intracellular cell permeability and down-regulating protein kinase Cζ activity to a functionally ineffective level down-regulates said intracellular endothelial cell permeability.

Still more preferably said endothelial cell is a vascular endothelial cell or a lymphatic endothelial cell.

Reference to “protein kinase Cζ” should be understood as a reference to all forms of this protein and to functional derivatives, homologues, analogues, chemical equivalents or mimetics thereof. This includes, for example, any isoforms which arise from alternative splicing of the subject protein kinase Cζ mRNA or mutants or polymorphic variants of these proteins.

Without limiting the present invention to any one theory or mode of action, thrombin signalling leads to, inter alia, increased intercellular vascular endothelial cell permeability. However, since the thrombin receptor PAR-1 activates a diverse range of downstream signalling intermediates, the precise signalling pathway involved in this mechanism had previously been obscure. It has now been determined that the atypical form of protein kinase C, being the protein kinase Cζ isoform, regulates vascular endothelial cell leakiness via the PI3-kinase dependent pathway. However, although the present invention has been exemplified with respect to thrombin, this should not be understood as a limitation with respect to the present invention which nevertheless encompasses modulation of endothelial cell permeability by any means including, but not limited to, via thrombin, VEGF and TNF stimulation. It has been determined that endothelial cell permeability, per se, is regulated via the protein kinase Cζ signalling pathway, irrespective of the nature of the specific stimulatory or inhibitory signal.

Reference to “modulating” should be understood as a reference to up-regulating or down-regulating the subject endothelial cell activity. Reference to “down-regulating” endothelial cell activity should therefore be understood as a reference to preventing, reducing (eg. slowing) or otherwise inhibiting one or more aspects of the functioning of the endothelial cell (for example retarding or preventing thrombin-induced vascular endothelial cell intercellular permeability) while reference to “up-regulating” should be understood to have the converse meaning.

Still without limiting the present invention in any way, thrombin is a highly pleiotropic molecule which exhibits multiple roles central to vascular biology including, but not limited to, acting upon platelets, endothelial cells and circulating clotting factors. Thrombin is a potent activator of vascular endothelial cells, increasing intercellular gap formation and permeability of confluent vascular endothelial cell monolayers. It is thought that the protein kinase Cζ mediated thrombin activity predominantly relates to increasing endothelial cell permeability (either intercellularly or intracellularly) and therefore regulating endothelial cell leakiness. Although the present invention extends to modulation of vascular permeability, per se, in a preferred embodiment, said vascular permeability is thrombin-induced permeability.

The present invention therefore most preferably provides a method of modulating thrombin induced intercellular endothelial cell permeability, said method comprising modulating the functional activity of protein kinase Cζ wherein up-regulating protein kinase Cζ activity to a functionally effective level up-regulates said permeability and down-regulating said protein kinase Cζ activity to a functionally ineffective level down-regulates said permeability.

More preferably, said endothelial cell is a vascular endothelial cell or a lymphatic endothelial cell.

Even more preferably, said permeability is either intercellular or intracellular.

By “thrombin” is meant all forms of thrombin and functional derivatives, homologues, analogues, chemical equivalents and mimetics thereof. Reference to “thrombin” should also be understood to include reference to any isoforms which arise from alternative splicing of thrombin mRNA or mutants or polymorphic variants of thrombin. It should also be understood to include reference to any other molecule which exhibits thrombin functional activity to the extent that the subject molecule mimics one or more thrombin signalling events by inducing signalling through a thrombin or thrombin-like receptor. Without limiting the present invention to any one theory or mode of action, thrombin signals via the protease activated receptor PAR-1. Protein kinase Cζ is activated via the PI3 kinase dependent pathway to induce vascular leakage. Since the method of the present invention is directed to modulating endothelial cell activity by modulating an intracellular signalling event which has been induced as a result of the interaction of thrombin with its receptor, this methodology can be applied to modulating such a cellular activity, irrespective of whether it has been induced by the interaction of thrombin with a PAR-1 receptor or the interaction of a thrombin mimetic, such as the naturally occurring or non-naturally occurring mimetic or analogue, with the subject receptor. It is conceivable, for example, that there may be naturally or non-naturally occurring thrombin mimetics (for example, toxins or drugs) which, if they were introduced into an individual, would induce unwanted thrombin-like endothelial cell activities due to their interaction with the PAR-1 receptor. Accordingly, the present invention should be understood to extend to the modulation of such cellular activities which are herein defined as falling within the scope of being “thrombin induced”. It should also be understood that to the extent that it may be desirable to up-regulate the cellular activity which is normally induced by thrombin, the method of the present invention provides a means of optionally circumventing the requirement for thrombin stimulation by directly up-regulating the protein kinase Cζ intracellular signalling pathway. It should be understood that this type of up-regulation nevertheless falls within the scope of a “thrombin-induced” activity since the method of the present invention effectively provides a means of mimicking this type of activity.

Reference to protein kinase Cζ “activity” should be understood as a reference to any one or more of the activities which protein kinase Cζ can perform. For example, and without limiting the present invention in any way, the activity of protein kinase Cζ is controlled by its phosphorylation status and relocalisation. Specifically, protein kinase Cζ is one of the substrates for phosphoinositide-dependent kinase-1 (PDK-1), a pivotal component of the phosphionositide 3 OH-kinase (PI3-kinase) signalling pathway. Subsequently to protein kinase Cζ activation, downstream signalling events include alteration of junctional molecules such as PECAM, VE cadherin, JAM-2 and catenins, which are all involved with regulation of junctional integrity in endothelial cells. Protein kinase Cζ is also thought to affect the actin cytoskeleton through modulation of Rho and rac. Accordingly, reference to “modulating” protein kinase Cζ functional activity is a reference to either up-regulating or down-regulating protein kinase Cζ functional activity. Such modulation may be achieved by any suitable means and includes:
(i) Modulating absolute levels of the active or inactive forms of protein kinase Cζ (for example increasing or decreasing intracellular protein kinase Cζ concentrations) such that either more or less protein kinase Cζ is available for activation and/or to interact with its downstream targets.
(ii) Agonising or antagonising protein kinase Cζ such that the functional effectiveness of any given protein kinase Cζ molecule is either increased, decreased or otherwise modulated or changed. For example, increasing the half life of protein kinase Cζ may achieve an increase in the overall level of protein kinase Cζ activity without actually necessitating an increase in the absolute intracellular concentration of protein kinase Cζ. Similarly, the partial antagonism of protein kinase Cζ, for example by coupling protein kinase Cζ to a molecule that introduces some steric hindrance in relation to the binding of protein kinase Cζ to its downstream targets, may act to reduce, although not necessarily eliminate, the effectiveness of protein kinase Cζ signalling. Accordingly, this may provide a means of down-regulating protein kinase Cζ functioning without necessarily down-regulating absolute concentrations of protein kinase Cζ.

In terms of achieving the up or down-regulation of protein kinase Cζ functioning, means for achieving this objective would be well known to the person of skill in the art and include, but are not limited to:
(i) Introducing into a cell a nucleic acid molecule encoding protein kinase Cζ or functional equivalent, derivative or analogue thereof in order to up-regulate the capacity of said cell to express protein kinase Cζ.
(ii) Introducing into a cell a proteinaceous or non-proteinaceous molecule which modulates transcriptional and/or translational regulation of a gene, wherein this gene may be a protein kinase Cζ gene or functional portion thereof or some other gene which directly or indirectly modulates the expression of the protein kinase Cζ gene.
(iii) Introducing into a cell the protein kinase Cζ expression product (in either active or inactive form) or a functional derivative, homologue, analogue, equivalent or mimetic thereof.
(iv) Introducing a proteinaceous or non-proteinaceous molecule which functions as an antagonist to the protein kinase Cζ expression product.
(v) Introducing a proteinaceous or non-proteinaceous molecule which functions as an agonist of the protein kinase Cζ expression product.
(vi) Introducing a proteinaceous or non-proteinaceous molecule which modulates the nature of the functional activity exhibited by protein kinase Cζ.

The proteinaceous molecules described above may be derived from any suitable source such as natural, recombinant or synthetic sources and includes fusion proteins or molecules which have been identified following, for example, natural product screening. The reference to non-proteinaceous molecules may be, for example, a reference to a nucleic acid molecule or it may be a molecule derived from natural sources, such as for example natural product screening, or may be a chemically synthesised molecule. The present invention contemplates analogues of the protein kinase Cζ expression product or small molecules capable of acting as agonists or antagonists. Chemical agonists may not necessarily be derived from the protein kinase Cζ expression product but may share certain conformational similarities. Alternatively, chemical agonists may be specifically designed to meet certain physiochemical properties. Antagonists may be any compound capable of blocking, inhibiting or otherwise preventing protein kinase Cζ from carrying out its normal biological function, such as molecules which prevent its activation or else prevent the downstream functioning of activated protein kinase Cζ. Antagonists include monoclonal antibodies and antisense nucleic acids which prevent transcription or translation of protein kinase Cζ genes or mRNA in mammalian cells. Modulation of expression may also be achieved utilising antigens, RNA, ribosomes, DNAzymes, RNA aptamers, antibodies or molecules suitable for use in cosuppression. The proteinaceous and non-proteinaceous molecules referred to in points (i)-(v), above, are herein collectively referred to as “modulatory agents”.

Screening for the modulatory agents hereinbefore defined can be achieved by any one of several suitable methods including, but in no way limited to, contacting a cell comprising the protein kinase Cζ gene or functional equivalent or derivative thereof with an agent and screening for the modulation of protein kinase Cζ protein production or functional activity, modulation of the expression of a nucleic acid molecule encoding protein kinase Cζ or modulation of the activity or expression of a downstream protein kinase Cζ cellular target. Detecting such modulation can be achieved utilising techniques such as Western blotting, electrophoretic mobility shift assays and/or the readout of reporters of protein kinase Cζ activity such as luciferases, CAT and the like.

It should be understood that the protein kinase Cζ gene or functional equivalent or derivative thereof may be naturally occurring in the cell which is the subject of testing or it may have been transfected into a host cell for the purpose of testing. Further, the naturally occurring or transfected gene may be constitutively expressed—thereby providing a model useful for, inter alia, screening for agents which down regulate protein kinase Cζ activity, at either the nucleic acid or expression product levels, or the gene may require activation—thereby providing a model useful for, inter alia, screening for agents which up regulate protein kinase Cζ expression. Further, to the extent that a protein kinase Cζ nucleic acid molecule is transfected into a cell, that molecule may comprise the entire protein kinase Cζ gene or it may merely comprise a portion of the gene such as the portion which regulates expression of the protein kinase Cζ product. For example, the protein kinase Cζ promoter region may be transfected into the cell which is the subject of testing. In this regard, where only the promoter is utilised, detecting modulation of the activity of the promoter can be achieved, for example, by ligating the promoter to a reporter gene. For example, the promoter may be ligated to luciferase or a CAT reporter, the modulation of expression of which gene can be detected via modulation of fluorescence intensity or CAT reporter activity, respectively.

In another example, the subject of detection could be a downstream protein kinase Cζ regulatory target, rather than protein kinase Cζ itself. Yet another example includes protein kinase Cζ binding sites ligated to a minimal reporter. For example, modulation of protein kinase Cζ activity can be detected by screening for the modulation of the functional activity in an endothelial cell. This is an example of an indirect system where modulation of protein kinase Cζ expression, per se, is not the subject of detection. Rather, modulation of the molecules which protein kinase Cζ regulates the expression of, are monitored.

These methods provide a mechanism for performing high throughput screening of putative modulatory agents such as the proteinaceous or non-proteinaceous agents comprising synthetic, combinatorial, chemical and natural libraries. These methods will also facilitate the detection of agents which bind either the protein kinase Cζ nucleic acid molecule or expression product itself or which modulate the expression of an upstream molecule, which upstream molecule subsequently modulates protein kinase Cζ expression or expression product activity. Accordingly, these methods provide a mechanism of detecting agents which either directly or indirectly modulate protein kinase Cζ expression and/or activity.

It should be understood that a related aspect of the present invention is directed to methods of screening for said modulatory agents.

The agents which are utilised in accordance with the method of the present invention may take any suitable form. For example, proteinaceous agents may be glycosylated or unglycosylated, phosphorylated or dephosphorylated to various degrees and/or may contain a range of other molecules used, linked, bound or otherwise associated with the proteins such as amino acids, lipid, carbohydrates or other peptides, polypeptides or proteins. Similarly, the subject non-proteinaceous molecules may also take any suitable form. Both the proteinaceous and non-proteinaceous agents herein described may be linked, bound otherwise associated with any other proteinaceous or non-proteinaceous molecules. For example, in one embodiment of the present invention, said agent is associated with a molecule which permits its targeting to a localised region.

The subject proteinaceous or non-proteinaceous molecule may act either directly or indirectly to modulate the expression of protein kinase Cζ or the activity of the protein kinase Cζ expression product. Said molecule acts directly if it associates with the protein kinase Cζ nucleic acid molecule or expression product to modulate expression or activity, respectively. Said molecule acts indirectly if it associates with a molecule other than the protein kinase Cζ nucleic acid molecule or expression product which other molecule either directly or indirectly modulates the expression or activity of the protein kinase Cζ nucleic acid molecule or expression product, respectively. Accordingly, the method of the present invention encompasses the regulation of protein kinase Cζ nucleic acid molecule expression or expression product activity via the induction of a cascade of regulatory steps.

The term “expression” refers to the transcription and translation of a nucleic acid molecule. Reference to “expression product” is a reference to the product produced from the transcription and translation of a nucleic acid molecule. Reference to “modulation” should be understood as a reference to up-regulation or down-regulation.

“Derivatives” of the molecules herein described (for example thrombin, protein kinase Cζ or other proteinaceous or non-proteinaceous agents) include fragments, parts, portions or variants from either natural or non-natural sources. Non-natural sources include, for example, recombinant or synthetic sources. By “recombinant sources” is meant that the cellular source from which the subject molecule is harvested has been genetically altered. This may occur, for example, in order to increase or otherwise enhance the rate and volume of production by that particular cellular source. Parts or fragments include, for example, active regions of the molecule. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterised by the removal of one or more amino acids from the sequence. Substitutional amino acid variants are those in which at least one residue in a sequence has been removed and a different residue inserted in its place. Additions to amino acid sequences include fusions with other peptides, polypeptides or proteins, as detailed above.

Derivatives also include fragments having particular epitopes or parts of the entire protein fused to peptides, polypeptides or other proteinaceous or non-proteinaceous molecules. For example, protein kinase Cζ or derivative thereof may be fused to a molecule to facilitate its entry into a cell. Analogs of the molecules contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogs.

Derivatives of nucleic acid sequences which may be utilised in accordance with the method of the present invention may similarly be derived from single or multiple nucleotide substitutions, deletions and/or additions including fusion with other nucleic acid molecules. The derivatives of the nucleic acid molecules utilised in the present invention include oligonucleotides, PCR primers, antisense molecules, molecules suitable for use in cosuppression and fusion of nucleic acid molecules. Derivatives of nucleic acid sequences also include degenerate variants.

A “variant” or “mutant” of thrombin or protein kinase Cζ should be understood to mean molecules which exhibit at least some of the functional activity of the form of thrombin or protein kinase Cζ of which it is a variant or mutant. A variation or mutation may tale any form and may be naturally or non-naturally occurring.

A “homologue” is meant that the molecule is derived from a species other than that which is being treated in accordance with the method of the present invention. This may occur, for example, where it is determined that a species other than that which is being treated produces a form of protein kinase Cζ which exhibits similar and suitable functional characteristics to that of the protein kinase Cζ which is naturally produced by the subject undergoing treatment.

Chemical and functional equivalents should be understood as molecules exhibiting any one or more of the functional activities of the subject molecule, which functional equivalents may be derived from any source such as being chemically synthesised or identified via screening processes such as natural product screening. For example chemical or functional equivalents can be designed and/or identified utilising well known methods such as combinatorial chemistry or high throughput screening of recombinant libraries or following natural product screening.

For example, libraries containing small organic molecules may be screened, wherein organic molecules having a large number of specific parent group substitutions are used. A general synthetic scheme may follow published methods (eg., Bunin B A, et al. (1994) Proc. Natl. Acad Sci. USA, 91:4708-4712; DeWitt S H, et al. (1993) Proc. Natl. Acad Sci. USA, 90:6909-6913). Briefly, at each successive synthetic step, one of a plurality of different selected substituents is added to each of a selected subset of tubes in an array, with the selection of tube subsets being such as to generate all possible permutation of the different substituents employed in producing the library. One suitable permutation strategy is outlined in U.S. Pat. No. 5,763,263.

There is currently widespread interest in using combinational libraries of random organic molecules to search for biologically active compounds (see for example U.S. Pat. No. 5,763,263). Ligands discovered by screening libraries of this type may be useful in mimicking or blocking natural ligands or interfering with the naturally occurring ligands of a biological target. In the present context, for example, they may be used as a starting point for developing protein kinase Cζ analogues which exhibit properties such as more potent pharmacological effects. Protein kinase Cζ or a functional part thereof may according to the present invention be used in combination libraries formed by various solid-phase or solution-phase synthetic methods (see for example U.S. Pat. No. 5,763,263 and references cited therein). By use of techniques, such as that disclosed in U.S. Pat. No. 5,753,187, millions of new chemical and/or biological compounds may be routinely screened in less than a few weeks. Of the large number of compounds identified, only those exhibiting appropriate biological activity are further analysed.

With respect to high throughput library screening methods, oligomeric or small-molecule library compounds capable of interacting specifically with a selected biological agent, such as a biomolecule, a macromolecule complex, or cell, are screened utilising a combinational library device which is easily chosen by the person of skill in the art from the range of well-known methods, such as those described above. In such a method, each member of the library is screened for its ability to interact specifically with the selected agent. In practising the method, a biological agent is drawn into compound-containing tubes and allowed to interact with the individual library compound in each tube. The interaction is designed to produce a detectable signal that can be used to monitor the presence of the desired interaction. Preferably, the biological agent is present in an aqueous solution and further conditions are adapted depending on the desired interaction. Detection may be performed for example by any well-known functional or non-functional based method for the detection of substances.

In addition to screening for molecules which mimic the activity of protein kinase Cζ, it may also be desirable to identify and utilise molecules which function agonistically or antagonistically to protein kinase Cζ in order to up or down-regulate the functional activity of protein kinase Cζ in relation to modulating endothelial cell growth. The use of such molecules is described in more detail below. To the extent that the subject molecule is proteinaceous, it may be derived, for example, from natural or recombinant sources including fusion proteins or following, for example, the screening methods described above. The non-proteinaceous molecule may be, for example, a chemical or synthetic molecule which has also been identified or generated in accordance with the methodology identified above. Accordingly, the present invention contemplates the use of chemical analogues of protein kinase Cζ capable of acting as agonists or antagonists. Chemical agonists may not necessarily be derived from protein kinase Cζ but may share certain conformational similarities. Alternatively, chemical agonists may be specifically designed to mimic certain physiochemical properties of protein kinase Cζ. Antagonists may be any compound capable of blocking, inhibiting or otherwise preventing protein kinase Cζ from carrying out its normal biological functions. Antagonists include monoclonal antibodies specific for protein kinase Cζ or parts of protein kinase Cζ.

Analogues of protein kinase Cζ or of protein kinase Cζ agonistic or antagonistic agents contemplated herein include, but are not limited to, modifications to side chains, incorporating unnatural amino acids and/or derivatives during peptide, polypeptide or protein synthesis and the use of crosslinkers and other methods which impose conformational constraints on the analogues. The specific form which such modifications can take will depend on whether the subject molecule is proteinaceous or non-proteinaceous. The nature and/or suitability of a particular modification can be routinely determined by the person of skill in the art.

For example, examples of side chain modifications contemplated by the present invention include modifications of amino groups such as by reductive alkylation by reaction with an aldehyde followed by reduction with NaBH4; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivatisation, for example, to a corresponding amide.

Sulphydryl groups may be modified by methods such as carboxymethylation with iodoacetic acid or iodoacetamide; perfornic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alklaline pH.

Tryptophan residues may be modified by, for example, oxidation with N-bromosuccinimide or allylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 1.

TABLE 1

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl--aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl-Nmbc | | | |
| ethylamino)cyclopropane | | | |

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

Reference herein to attaining either a “functionally effective level” or “functionally ineffective level” of protein kinase Cζ should be understood as a reference to attaining that level of functionally active protein kinase Cζ at which modulation of endothelial cell activity can be achieved, whether that be upregulation or down-regulation. In this regard, it is within the skill of the person of skill in the art to determine, utilising routine procedures, the threshold level of functionally active protein kinase Cζ expression above which endothelial cell activity can be upregulated and below which endothelial cell activity is downregulated. For example, suitable for use in this regard is any method which regulates the phosphorylation status or the cellular localisation of protein kinase Cζ, as would any method which is based on the alteration of RNA synthesis of protein kinase Cζ (for example, antisense constructs, DNAzymes or RNA could change the levels of proteins). It should be understood that reference to an “effective level” means the level necessary to at least partly attain the desired response. The amount will vary depending on the health and physical condition of the cellular population and/or individual being treated, the taxonomic group of the cellular population and/or individual being treated, the degree of up or down-regulation which is desired, the formulation of the composition which is utilised, the assessment of the medical situation and other relevant factors. Accordingly, it is expected that this level may vary between individual situations, thereby falling in a broad range, which can be determined through routine trials.

The method of the present invention contemplates the modulation of endothelial cell functioning both in vitro and in vivo. Although the preferred method is to treat an individual in vivo it should nevertheless be understood that it may be desirable that the method of the invention may be applied in an in vitro environment, for example to provide an in vitro model of endothelial cell permeability analysis. In another example the application of the method of the present invention in an in vitro environment may extend to providing a read out mechanism for screening technologies such as those hereinbefore described. That is, molecules identified utilising these screening techniques can be assayed to observe the extent and/or nature of their functional effect on endothelial cells which have been functionally modulated according to the method of the present invention.

Although the preferred method is to down-regulate, in particular, thrombin-induced vascular endothelial cell activity, thereby essentially down-regulating intercellular vascular endothelial cell permeability (for example in order to down-regulate the progression of an inflammatory response or the development of a tumour), it should be understood that there may also be circumstances in which it is desirable to up-regulate intercellular vascular endothelial cell permeability. For example, and without limiting the present invention in any way, it has been observed that newly formed micro-vessels in tumours are highly permeable. This hyper-permeability allows the deposition of fibrin in tumours that supports and promotes adhesion and migration, being essential steps in the angiogenic response. To the extent that this occurs in tumours, it is clearly an object of the present invention to down-regulate the instance of such vascular hyper-permeability. However, in the context of organ or limb transplantation, up-regulating vascular hyper-permeability in order to facilitate angiogenesis in the new organ or limb may be highly desirable. In another example, an increase in vascular hyper-permeability may be desirable in terms of facilitating the local or systemic delivery of a drug.

Accordingly, another aspect of the present invention is directed to a method of regulating endothelial cell activity in a mammal, said method comprising modulating the functional activity of protein kinase Cζ in said mammal wherein up-regulating protein kinase Cζ activity to a functionally effective level up-regulates said endothelial cell activity and down-regulating protein kinase Cζ activity to a functionally ineffective level down-regulates said endothelial cell activity.

Preferably, said endothelial cell is a vascular endothelial cell or a lymphatic endothelial cell and said activity is intercellular endothelial cell permeability or intracellular endothelial cell permeability. Even more preferably, said activity is thrombin-induced vascular endothelial cell permeability.

Modulation of said protein kinase Cζ functional activity is achieved via the administration of protein kinase Cζ, a nucleic acid molecule encoding protein kinase Cζ or an agent which effects modulation of protein kinase Cζ activity or protein kinase Cζ gene expression (herein collectively referred to as “modulatory agents”). As detailed hereinbefore, it should be understood that the method of the present invention may be effected either with or without the presence of thrombin. In particular, and as detailed hereinbefore, the determination of the intracellular signalling mechanism which is utilised by thrombin in order to up-regulate endothelial cell activity now provides a means of modulating said activity either as a consequence of thrombin stimulation or as a means of circumventing the requirement for thrombin stimulation (this latter outcome is particularly useful in terms of the up-regulation of intercellular vascular endothelial cell permeability in the absence of thrombin stimulation).

Accordingly, in one preferred embodiment there is provided the method of up-regulating endothelial cell activity in a mammal, said method comprising administering to said mammal an effective amount of an agent for a time and under conditions sufficient to induce a functionally effective level of protein kinase Cζ.

In another preferred embodiment there is provided a method of up-regulating endothelial cell activity in a mammal, said method comprising administering to said mammal an effective amount of protein kinase Cζ for a time and under conditions sufficient to induce a functionally effective level of protein kinase Cζ.

In still another preferred embodiment there is provided a method of up-regulating endothelial cell activity in a mammal, said method comprising administering to said mammal an effective amount of a nucleotide sequence encoding protein kinase Cζ for a time and under conditions sufficient to induce a functionally effective level of protein kinase Cζ.

In yet another preferred embodiment there is provided a method of down-regulating endothelial cell activity in a mammal, said method comprising administering to said mammal an effective amount of an agent for a time and under conditions sufficient to induce a functionally ineffective level of protein kinase Cζ.

In accordance with these preferred embodiments of the present invention, said endothelial cell is preferably a vascular or lymphatic endothelial cell and said activity is preferably intercellular or intracellular endothelial cell permeability. Still more preferably, said activity is thrombin-induced vascular endothelial cell permeability.

Reference to “induce” should be understood as a reference to achieving the desired protein kinase Cζ level, whether that be a functionally effective level or a functionally ineffective level. Said induction is most likely to be achieved via the up-regulation or down-regulation of protein kinase Cζ functional activity, as hereinbefore described, although any other suitable means of achieving induction are nevertheless herewith encompassed by the method of the present invention.

A further aspect of the present invention relates to the use of the invention in relation to the treatment and/or prophylaxis of disease conditions or other unwanted conditions. Without limiting the present invention to any one theory or mode of action, the regulation of endothelial cell activity, and in particular intercellular vascular endothelial cell permeability, is an essential requirement in terms of controlling the passage of solutes and cellular from the circulation to the tissues both in terms of normal physiology and in the context of many unwanted pathologies. For example, newly formed micro-vessels and tumours are highly permeable, thereby facilitating the deposition of fibrin in tumours which supports and promotes cell adhesion and migration, essential steps in the angiogenic response. In another example, chronic inflammatory states such as rheumatoid arthritis and atherosclerosis are characterised by vessel hyper-permeability which allows increased transmigration of inflammatory cells across the activated endothelium. A number of factors have been described which promote this endothelial cell leakiness, including the activity of thrombin. Without limiting the present invention to any one theory or mode of action, it is thought that thrombin, for example, acts by inducing changes in junction or molecules such as PECAN-1 and VE-cadherin or their associated signalling molecules, such as the cateinins. Accordingly, the present invention is particularly useful, but in no way limited to, use as a therapy to down-regulate intercellular vascular endothelial cell permeability where an individual is suffering from an unwanted inflammatory condition or tumour development. Alternatively, the up-regulation of intercellular vascular endothelial cell permeability may be desirable where it is necessary that passage of solutes or cells is facilitated from the circulation into the tissue, such as for the purpose of facilitating angiogenesis in transplanted organs or limbs or in tissues which are subject to amyloid plaque deposition.

The present invention therefore contemplates a method for the treatment and/or prophylaxis of a condition characterised by aberrant, unwanted or otherwise inappropriate endothelial cell activity in a mammal, said method comprising modulating the functional activity of protein kinase Cζ wherein up-regulating protein kinase Cζ activity to a functionally effective level up-regulates said endothetial cell activity and down-regulating protein kinase Cζ activity to a functionally ineffective level down-regulates said cellular activity.

Preferably, said endothelial cells are vascular endothelial cells or lymphatic endothelial cells and said endothelial cell activity is intercellular permeability or intracellular permeability.

Reference to “aberrant, unwanted or otherwise inappropriate” endothelial cell functioning should be understood as a reference to under-active functioning, to physiologically normal functioning which is inappropriate in that it is unwanted or to over-active endothelial cell functioning. As detailed hereinbefore, there are a number of conditions which are dependent on the induction of the correct level of endothelial cell functioning, and in particular vascular endothelial cell functioning. For instance, and in relation to the preferred embodiments disclosed herein, in individuals experiencing an unwanted inflammatory response, the down-regulation of protein kinase Cζ to a functionally ineffective level provides a means for this unwanted inflammatory response to be retarded. In another example the development of new blood vessels is a process which is central to the progress and/or continuance of many disease conditions. For example, angiogenesis dependant diseases include, but are not limited to, solid tumors, blood born tumors such as leulkemias, tumor metastasis, benign tumors, for example hemangiomas, angiofibromas, acoustic neuromas, neurofibroms, trachomas, and pyogenic granulomas, rheumatoid arthritis, Crohn's disease, atherosclerosis, obesity, endometriosis, ocular angiogenic diseases, for example, diabetic retinopathy, retinopathy of prematurity (retrolental fibroplasia), macular degeneration, corneal graft rejection, rubeosis, and neovascular glaucoma; psoriasis, facial and truncal telangiectasias, Osler-Webber Rendau syndrome (hereditary hemorrhagic telangiectasia).

In a most preferred embodiment, there is provided a method for the treatment and/or prophylaxis of a condition characterised by unwanted endothelial cell activity in a mammal, said method comprising administering to said mammal an effective amount of an agent for a time and under conditions sufficient to induce a functionally ineffective level of protein kinase Cζ.

Preferably, endothelial cell is a vascular or lymphatic endothelial cell and said activity is endothelial cell permeability. Most preferably, said condition is an inflammatory response or a solid tumour.

An “effective amount” means an amount necessary at least partly to attain the desired response, or to delay the onset or inhibit progression or halt altogether, the onset or progression of the particular condition being treated. The amount varies depending upon the health and physical condition of the individual to be treated, the taxonomic group of the individual to be treated, the degree of protection desired, the formulation of the composition, the assessment of the medical situation, and other relevant factors. It is expected that the amount will fall in a relatively broad range that can be determined through routine trials.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

The present invention further contemplates a combination of therapies, such as the administration of the modulatory agent together with other proteinaceous or non-proteinaceous molecules which may facilitate the desired therapeutic or prophylactic outcome.

Administration of molecules of the present invention hereinbefore described [herein collectively referred to as “modulatory agent”], in the form of a pharmaceutical composition, may be performed by any convenient means. The modulatory agent of the pharmaceutical composition is contemplated to exhibit therapeutic activity when administered in an amount which depends on the particular case. The variation depends, for example, on the human or animal and the modulatory agent chosen. A broad range of doses may be applicable. Considering a patient, for example, from about 0.1 mg to about 1 mg of modulatory agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The modulatory agent may be administered in a convenient manner such as by the oral, intravenous (where water soluble), intraperitoneal, intramuscular, subcutaneous, intradermal or suppository routes or implanting (e.g. using slow release molecules). The modulatory agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip patch and implant. Preferably, said route of administration is oral.

In accordance with these methods, the agent defined in accordance with the present invention may be coadministered with one or more other compounds or molecules. By “coadministered” is meant simultaneous administration in the same formulation or in two different formulations via the same or different routes or sequential administration by the same or different routes. For example, the subject protein kinase Cζ may be administered together with an agonistic agent in order to enhance its effects. Alternatively, in the case of autoimmune inflanmmation, the protein kinase Cζ antagonist may be administered together with immunosuppressive drugs. By “sequential” administration is meant a time difference of from seconds, minutes, hours or days between the administration of the two types of molecules. These molecules may be administered in any order.

Another aspect of the present invention relates to the use of an agent capable of modulating the functionally effective level of protein kinase Cζ in the manufacture of a medicament for the regulation of endothelial cell activity in a mammal wherein up-regulating protein kinase Cζ activity to a functionally effective level up-regulates said endothelial cell activity and down-regulating protein kinase Cζ activity to a functionally ineffective level down-regulates said endothelial cell activity.

In another aspect the present invention relates to the use of protein kinase Cζ or a nucleic acid encoding protein kinase Cζ in the manufacture of a medicament for the regulation of endothelial cell activity wherein up-regulating protein kinase Cζ to a functionally level up-regulates said endothelial cell activity.

According to these preferred embodiments, the subject endothelial cells are preferably vascular or lymphatic endothelial cells. Still more preferably the subject endothelial cell functioning is intercellular or intracellular endothelial cell permeability. Most preferably, said functioning is down-regulated.

The term “mammal” and “subject” as used herein includes humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Preferably, the mammal is human or a laboratory test animal Even more preferably, the mammal is a human.

In yet another further aspect, the present invention contemplates a pharmaceutical composition comprising the modulatory agent as hereinbefore defined and one or more pharmaceutically acceptable carriers and/or diluents. Said agents are referred to as the active ingredients

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding protein kinase Cζ or a modulatory agent as hereinbefore defined. The vector may, for example, be a viral vector.

The present invention is further defined by the following non-limiting Examples.

### EXAMPLE 1

### Angiopoietin-1 Inhibits Thrombin Induced Endothelial Cell Permeability Changes by inhibition of Protien Kinase Cζ

### Materials and Methods

Reagents

Angiopoietin-1, obtained from Regeneron Inc was used as described (Gamble, J. R., Drew, J., Trezise, L., Underwood, A., Parsons, M., Kasminkas, L., Rudge, J., Yancopoulos, G., Vadas, M. A. (2000) Circ Res. 87:603-7; Papapetropoulos, A., Garcia-Cardena, G., Dengler, T. J., Maisonpierre, P. C., Yancopoulos, G. D. and Sessa, W. C. (1999) Lab Invest. 70:213-23). Bisindolylmaleimide I hydrochloride, Calphostin C and LY294002 were from Calbiochem Novabiochem, (Croydon, Vic, Australia). Chelerythrine chloride and H-89 were from Biomol Research Laboratories, Inc (Plymouth Meeting, Pa., USA). Thrombin, protease inhibitor cocktail and FITC-conjugated dextran were from Sigma-Aldrich (St. Louis, Mo., U.S.A)

Cells and Cell Culture

Human umbilical vein endothelial cells were grown in M119 medium with 20% foetal calf serum (FCS) as described (Litwin, M., Clark, K., Noack, L., Furze, J., Berndt, M., Albelda, S., Vadas, M., Gamble, J. (1997) J Cell Biol. 139:219-28). Cells were used at passage 4 or less. For infection with adenoviral constructs, cells were grown to 80% confluence and exposed to 2.5×10⁶ plaque forming units/25 cm² culture area for 2 hours in M119 medium with 2% FCS and a further 22 hours with 20% FCS.

Recombinant Adenoviral Constructs

DNA constructs encoding FLAG-protein kinase Cζ, and FLAG-protein kinase Cζ T410A, in pCMV5, and Myr-protein kinase Cζ-FLAG in pCMV6 were a generous gift from Dr. Alex Toker, Biomedical Research Institute, Boston, Mass., USA. Recombinant adenoviruses were constructed by subcloning EcoR1 fragments from the pCMV5 constructs and HindIII/EcoRI fragment from the pCMV6 construct into the pAdEasy-1 vector (Qbiogene Inc., Carlsbad, Calif., USA). Virus was amplified in HEK293 cells and purified by CsC1 gradient ultracentifugation. Virus titres were determined using the TCID₅₀ method as recommended by Qbiogene.

Endothelial Permeability Assays

Assays were performed as described (Draijer et al. (1995) supra). Endothelial cells (10⁵) were cultured in transwells (3 μ-pore size, Corning Costar Corp, Cambridge, Mass., USA) for 24 hours in complete medium and then in 2% FCS medium for a further 24 hours. Cells were pre-treated with either bisindolylmaleimide I (10 nM or 6 μM), Calphostin C (100 nM), chelerythrine chloride (1 μM), H-89 (50 nM) or angiopoietin-1 (0.1 μg/ml), as required. FITC-conjugated dextran (0.2 μg) was added to the upper chamber of all wells and cells were then treated with thrombin (0.2 U/ml). The relative fluorescence in the lower chambers of the transwells was determined using a LS 50B Luminescence Spectrometer (Perkin Elmer, Beaconsfield, Buckinghamshire, UK; excitation wavelength, 485 nm; emission wavelength 530 nm) after 30 minutes treatment. Adenovirus infected ECs were plated on to transwells 24 hours after the above infection procedure and treated in the same manner as uninfected cells.

Immunoblotting

Endothelial cells were plated into fibronectin coated flasks before infection as above. Following infection, cells were serum depleted (0.2% FCS) overnight. Cells were then treated with angiopoietin-1, thrombin and/or bisindolylmaleimide I, as required, lysed in ice-cold lysis buffer (50 mM Tris.HCl, pH 7.4, with 1% NP-40, 150 mM NaCl, 2 mM EGTA, 1 mM NaPO4, 100 mM NaF, 10 mM sodium pyrophosphate and protease inhibitor cocktail). Protein concentrations were assayed using Bradford Reagent (BioRad, Hercules, Calif., USA). Equal amounts of protein were loaded onto 10% acrylamide gels, separated by SDS-PAGE, transferred to PVDF membrane, blocked with 5% skim milk powder and 0.1% Triton-X100 in phosphate buffered saline, and probed with a polyclonal rabbit antibody directed against phosphorylated Thr410 of the activation loop of protein kinase Cζ (Chou, M. M., Hou, W., Johnson, J., Graham, L. K., Lee, M. H., Chen, C. S., Newton, A. C., Schaffhausen, B. S., Toker, A. (1998) Curr Biol. 8:1069-77). This antibody was a generous gift from Dr Alex Toker. After washing, membranes were incubated with anti-rabbit secondary antibody and reactive bands were detected by chemiluminescence (ECL Western Blotting Detection Reagents, Amersham Pharmacia Biotech, Little Chalfont, England, UK). Membranes were stripped using stripping buffer (Re-Blot Plus Western Blot Recycling Kit, Chemicon, Temecula Calif., USA) and re-probed with rabbit anti-protein kinase Cζ immunoaffinity purified IgG (Upstate Biotechnology, Lake Placid N.Y., USA).

Immunofluorescence

6×10⁴ endothelial cells were cultured in fibronectin coated glass LabTek chamber slides (Nalge Nunc International, Naperville, Ill., USA) and incubated for 3 days prior to staining. Cells were then treated with angiopoietin-1, thrombin and/or bisindolylmaleimnide I, as required. Cells were washed once in PBS, fixed in 4% paraformnaldehyde/PBS for 5 minutes, permeabilized with acetone for 5 min at −20° C. and then washed twice with PBS. The fixed cells were incubated with rabbit anti-protein kinase Cζ immunoaffmity purified IgG or anti-protein kinase Cλ (BD Transduction Laboratories, San Diego, Calif., USA) overnight at 4° C. followed by FITC-conjugated anti-rabbit antibody (Rockland, Gilbertsville Pa., USA). Coverslips were mounted using fluorescent microscopy mounting medium (Dako Corp., Carpinteria, Calif., USA). Cells were imaged by epifluorescent microscopy on an Olympus BX-51 microscope (Olympus, Hamburg, Germany) equipped with excitation filters for fluorescein (494 nm) acquired to a Photometrics Cool Snap FX charge-coupled device camera (Roper Scientific GmbH, Germany). Images were adjusted for brightness and contrast using V⁺⁺ software (Digital Optics Ltd., Aucldand, New Zealand).

### EXAMPLE 2

### Thrombin-Induced Increased in EC Permeability are Inhibited by Blocking Protein Kinease Cζ Signalling

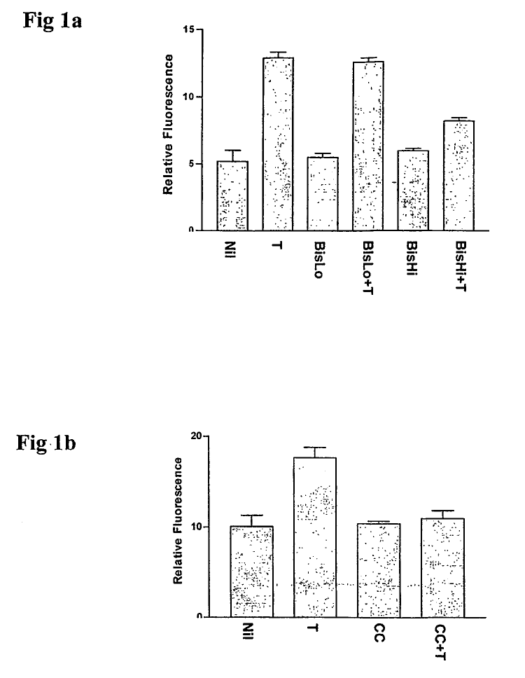

Thrombin signaling in vascular EC is mediated by the protease-activated receptor PAR-1 and protein kinase Cs are down-stream targets of PAR1 (Coughlin, S. R. (2000) Nature 407:258-64). To clarify the role of protein kinase C and the specific isoforms in thrombin-induced permeability, we took advantage of the varying specificity of protein Icinase C inhibitors. We used Chelerythrine Chloride, an inhibitor of all protein Icinase C isoforms (Laudanna, C., Mochly-Rosen, D., Liron, T., Constantin, G., Butcher, E. C. (1998) J Biol Chem. 273:30306-15), Calphostin C, an inhibitor of classical and novel protein kinase Cs (Kobayashi, E., Nakano, H., Morimoto, M., Tamaoki, T. (1989) Biochem Biophys Res Commun. 159:548-53), and bisindolylmaleimide I, a concentration dependent inhibitor of protein kinase C (Martiny-Baron, G., Kazanietz, M. G., Mischak, H., Blumberg, P. M., Kochs, G., Hug, H., Marme, D., Schachtele, C. (1993) J Biol Chem. 268:9194-7), whereby at 100 nM it inhibits classical and novel protein kinase C isoforms while at 6 μM it inhibits all protein cinase C isoforms (Martiny-Baron et al. (1993) supra; Uberall, F., Hellbert, K., Kampfer, S., Maly, K., Villunger, A., Spitaler, M., Mwanjewe, J., Baier-Bitterlich, G., Baier, G., Grunicke, H. H. (1999) J Cell Biol. 144:413-25). Using the passage of FITC labelled dextran through monolayers of human umbilical vein endothelial cells as a measure of permeability we found that both bisindolylmaleimide I at 6 μM (FIG. 1a) and chelerythrine chloride at 1 μM (FIG. 1b) inhibited thrombin stimulation of endothelial cell permeability supporting the involvement of protein kinase C. High concentrations of bisindolylmaleimide I can also inhibit protein kinase A (PKA), however, the specific protein kinase A inhibitor H-89 has no effect on thrombin induced permeability changes (FIG. 1c). Calphostin C and bisindolylmaleimide I at 100 nM had no effect on the thrombin-induced increases of endothelial cell permeability (FIG. 1d, 1a). Together the use of these inhibitors suggested that classical and novel protein kinase Cs are not involved but atypical protein kinase C isoforms are central to thrombin-induced endothelial cell permeability increases.

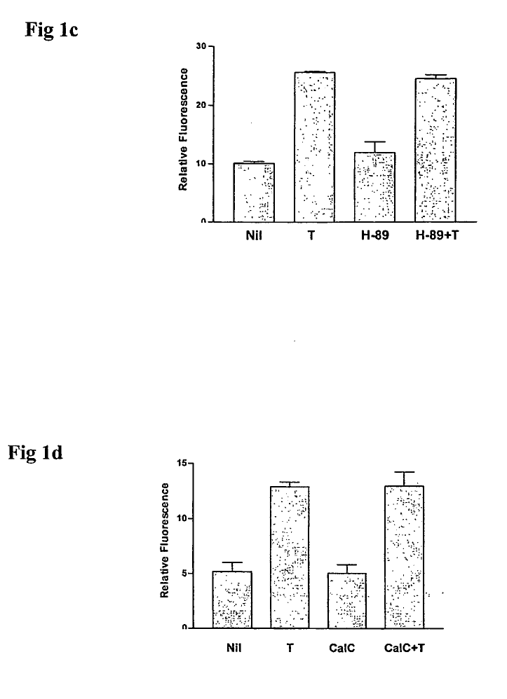

The two atypical protein kinase C isoforms protein kinase Cζ and protein kinase Cλ are present in endothelial cells (Li, H., Oehrlein, S. A., Wallerath, T., Ihrig-Biedert, I., Wohlfart, P., UIlshofer, T., Jessen, T., Herget, T., Forstermann, U., Kleinert, H. (1998) Mol Pharmacol 53:630-7). To determine if one or both of these isoforms are involved in thrombin-mediated permeability changes, confluent quiescent endothelial cells were stained with anti-protein kinase Cζ and anti-protein kinase Cλ antibodies. Both protein kinase Cζ and protein kinase Cλ were distributed throughout the cytoplasm (FIG. 2b, 2e). When confluent monolayers of endothelial cell were treated with thrombin, protein kinase Cζ localized to the cell membrane (FIG. 2c) while protein Icinase Cλ remained evenly distributed despite significant contraction of individual cells, characteristic of the response to thrombin stimulation (FIG. 2f). As targeting of protein kinase C isoforms to the plasma membrane indicates enzyme activation Nishizuka, Y. (2001) Alcohol Clin Exp Res. 25:3S-7S), this observation is consistent with thrombin stimulation activating protein kinase Cζ but not protein kinase Cλ.

### EXAMPLE 3

### Activation of Protein Kinase Cζ Increases the Permeability of Endothelial Cell

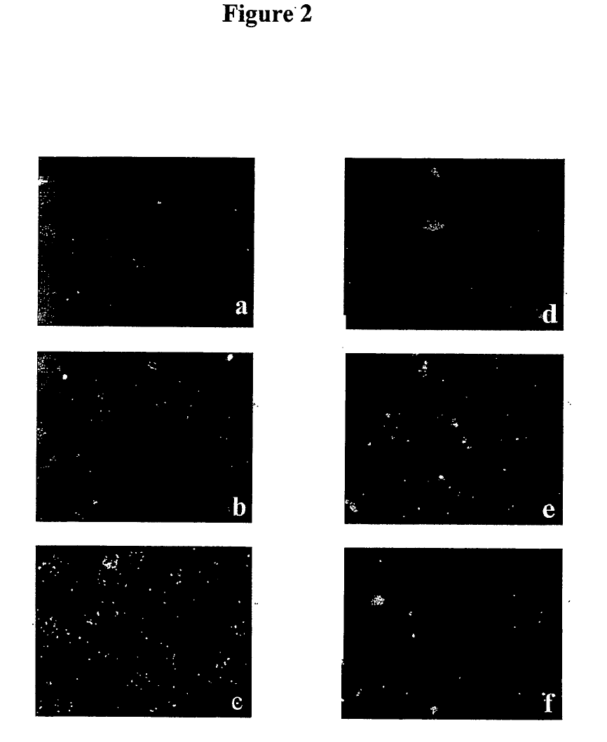

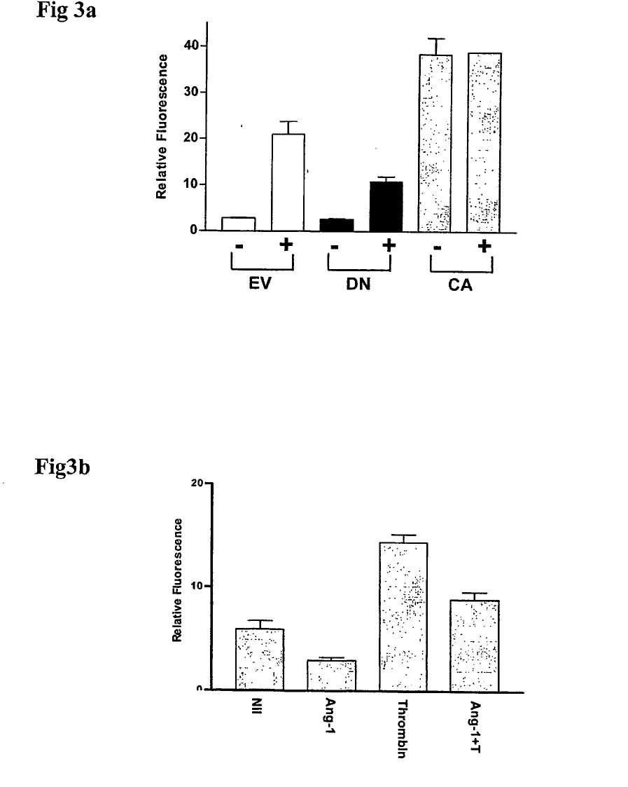

To confirm that protein kinase Cζ is a mediator of thrombin stimulated increases in endothelial cell permeability, wild-type, dominant-negative and constitutively active protein kinase Cζ were over-expressed in endothelial cell by infection with adenovirus carrying these constructs. Dominant-negative protein kinase Cζ has the critical threonine of the activation-loop, at position 410, mutated to alanine (Chou et al. (1998) supra). Constitutively active protein kinase Cζ results from fusion of the amino-terminal myristoylation sequence of p60 c-Src to the amino terminus of protein kinase Cζ, thereby constitutively targeting the resultant protein to the cell membrane (Chou et al. (1998) supra). Cells over-expressing wild-type protein kinase Cζ responded to thrombin in a similar way to cells infected with empty vector (data not shown). Infection of endothelial cell with adenovirus carrying the dominant-negative protein kinase Cζ resulted in inhibition of the thrombin stimulated increase in endothelial cell permeability when compared with cells infected with empty vector (FIG. 3a) or wild-type protein kinase Cζ (data not shown). Conversely, endothelial cells infected with adenovirus carrying the constitutively active protein kinase Cζ were highly permeable, even in the absence of thrombin, and thrombin stimulation had no further effect on permeability. Thus, protein Icinase Cζ appears to be critical in maintaining endothelial cell junction integrity and is implicated in thrombin induced endothelial cell permeability increases.

### EXAMPLE 4

### Angiopoietin-1 Inhibits Thrombin-Induced Relocalisation and Phosphorylation of Protein Minase Cζ in Endothelial Cell

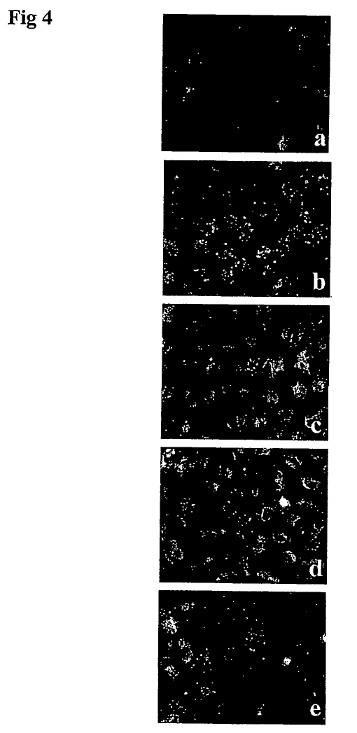

Angiopoietin-1 can inhibit the thrombin-induced increase of endothelial cell permeability (Gamble et al. (2000) supra) (FIG. 3b). We therefore investigated whether the angiopoietin-1 mediated inhibition was through regulation of protein kinase Cζ. Staining of confluent quiescent endothelial cell with the anti-protein kinase Cζ antibody showed protein kinase Cζ to be distributed throughout the cytoplasm (FIG. 2b and FIG. 4a). Thrombin stimulation caused protein kinase Cζ to be localized to the cell membrane (FIG. 2c and FIG. 4b). Concurrently, the shape of the endothelial cells changed with progressive retraction of the membrane and gaps between cells becoming evident. When endothelial cells were pre-treated with angiopoietin-1, at 0.1 μg/ml for 30 minutes, or bisindolylmaleimide I, 6 μM for 15 minutes, followed by thrombin treatment (FIG. 4c, 4d), protein cinase Cζ localization to the membrane was dramatically decreased thus indicating that angiopoietin-1 inhibition of thrombin stimulation may occur through inhibition of protein kinase Cζ translocation and activation.

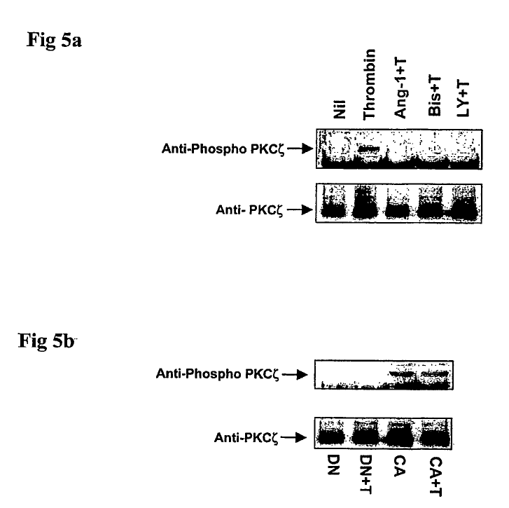

Phosphorylation of the threonine in the activation-loop of protein kinase Cs is the critical first step of PKC activation. This is rapidly followed by autophosphorylation of a 30 threonine and a serine residue within the catalytic domain and concomitant translocation from the cytoplasm to the cell membrane (Parekh et al. (2000) supra; Chou et al. (1998) supra). Phosphorylated protein kinase CCζ could not be detected using an antibody specific for the phosphorylated Thr 410 within the activation loop of protein kinase C following thrombin treatment of normal endothelial cells, presumably because the levels of the enzyme are low. Protein kinase Cζ was then over-expressed by adenoviral mediated infection. Thrombin treatment for 15 minutes increased the phosphorylation of over-expressed protein kinase Cζ (FIG. 5a) while pretreatment with angiopoietin-1 at 0.1 μg/ml for 30 minutes or bisindolylmaleimide I at 6 μM for 15 minutes inhibited thrombin stimulated protein kinase Cζ phosphorylation. As expected, endothelial cells infected with dominant-negative protein kinase Cζ showed no protein Icinase Cζ phosphorylation in response to thrombin while those expressing constitutively-active protein kinase Cζ showed constitutive protein kinase Cζ phosphorylation (FIG. 5b).

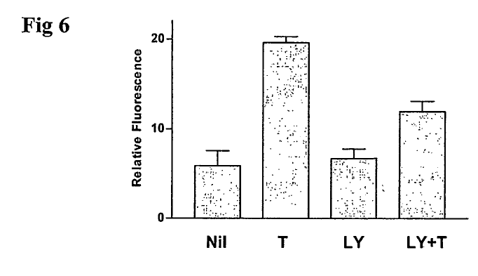

Receptor-mediated activation of protein kinase Cζ may occur via phosphatidylinositol-3-kinase (PI3-kinase) activation (Parekh et al. (2000) supra; Chou et al. (1998) supra; Le Good, J. A., Ziegler, W. H., Parekh, D. B., Alessi, D. R., Cohen, P., Parker, P. J. (1998) Science 281:2042-5). Indeed this appears to be the case in these cells as pre-treatment of endothelial cells with the PI3-kinase inhibitor LY294002 inhibited thrombin-induced permeability increases (FIG. 6) and blocked both protein kinase Cζ relocalisation (FIG. 4e) and phosphorylation (FIG. 5a) in response to thrombin. This is consistent with thrombin activating a PI3-kinase-dependent pathway resulting in protein kinase Cζ activation, leading to increases in endothelial cell permeability.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

(Dvorak, H. F., Harvey, V. S., Estrella, P., Brown, L. F., McDonagh, J., Dvorak, A. M. (1987) Lab Invest. 57:673-86.
Bunin B A, et al. (1994) Proc. Natl. Acad. Sci. USA, 91:4708-4712.
Bussolino F, Silvagno F, Garbarino G, Costamagna C, Sanavio F, Arese M, Soldi R, Aglietta M, Pescarmona G, Camussi G, et al. (1994) J Biol Chem. 269:2877-86.
Chou, M. M., Hou, W., Johnson, J., Graham, L. K., Lee, M. H., Chen, C. S., Newton, A. C., Schaffhausen, B. S., Toker, A. (1998) Curr Biol. 8:1069-77.
Coughlin, S. R. (2000) Nature 407:258-64.
DeWitt S H, et al. (1993) Proc. Natl. Acad. Sci. USA, 90:6909-6913.
Draijer, R., Atsma, D. E., van der Laarse, A., van Hinsberg, V. W. (1995) Circ Res. 76:199-208.
Dvoral, H. F., Brown, L. F., Detmar, M., Dvorak, A. M. (1995) Am J Pathol. 146:1029-39.
Gamble, J. R., Drew, J., Trezise, L., Underwood, A., Parsons, M., Kasminkas, L., Rudge, J., Yancopoulos, G., Vadas, M. A. (2000) Circ Res. 87:603-7.
Garcia, J. G., Verin, A. D., Schaphorst, K. L. (1996) Semin Thromb Hemost. 22:309-15.
Hempel, 1997
Kobayashi, E., Nakano, H., Morimoto, M., Tamaoki, T. (1989) Biochem Biophys Res Commun. 159:548-53.
Laudanna, C., Mochly-Rosen, D., Liron, T., Constantin, G., Butcher, E. C. (1998) J Biol Chem. 273:30306-15.
Le Good, J. A., Ziegler, W. H., Parekh, D. B., Alessi, D. R., Cohen, P., Parker, P. J. (1998) Science 281:2042-5.
Li, H., Oehrlein, S. A., Wallerath, T., Ihrig-Biedert, I., Wohlfart, P., Ulshofer, T., Jessen, T., Herget, T., Forstermann, U., Kleinert, H. (1998) Mol Pharmacol. 53:630-7).
Litwin, M., Clark, K., Noack, L., Furze, J., Bemdt, M., Albelda, S., Vadas, M., Gamble, J. (1997) J Cell Biol. 139:219-28.
Lum, H., Andersen, T. T., Siflinger-Birnboim, A., Tiruppathi, C., Goligorsky, M. S., Fenton, J. W. 2nd, Malik, A. B. (1993) J Cell Biol. 120:1491-9.
Lynch J J, Ferro T J, Blumenstock F A, Brockenauer A M, Malilc A B. (1990) J Clin Invest. 85:1991-8.
Macfarlane, S. R., Seatter, M. J., Kanke, T., Hunter, G. D., Plevin, R. (2001) Pharmacol Rev. 53:245-82.
Malik, 1994
Martiny-Baron, G., Kazanietz, M. G., Mischak, H., Blumberg, P. M., Kochs, G., Hug, H., Marme, D., Schachtele, C. (1993) J Biol Chem. 268:9194-7.
Nishizuka, Y. (2001) Alcohol Clin Exp Res. 25:3S-7S.
Papapetropoulos, A., Garcia-Cardena, G., Dengler, T. J., Maisonpierre, P. C., Yancopoulos, G. D. and Sessa, W. C. (1999) Lab Invest. 70:213-23.
Parekh, D. B., Ziegler, W., Parker, P. J. (2000) Embo J 19:496-503.
Uberall, F., Hellbert, K., Kampfer, S., Maly, K., Villunger, A., Spitaler, M., Mwanjewe, J., Baier-Bitterlich, G., Baier, G., Grunicke, H. H. (1999) J Cell Biol. 144:413-25.
van Nieuw Amerongen G P, Draijer R, Vermeer M A, van Hinsbergh V W. (1998) Circ Res. 83:1115-23.
Vouret-Craviari V, Boquet P, Pouyssegur J, Van Obberghen-Schilling E.(1998) Mol Biol Cell. 9:2639-53.
Yamada Y, Furumichi T, Furui H, Yokoi T, Ito T, Yamauchi K, Yokota M, Hayashi H, Saito H. (1990) Arteriosclerosis 10:410-20.
Zhou G, Wooten M W, Coleman E S. (1994) Exp Cell Res. 214:1-11.

1. A method of modulating endothelial cell activity, said method comprising modulating the functional activity of protein Cζ, wherein up-regulating protein kinase Cζ activity to a functionally effective level up-regulates said cellular activity and down-regulating protein kinase Cζ activity to a functionally ineffective level down-regulates said cellular activity.

2. The method according to claim 1, wherein said endothelial cell is a vascular endothelial cell or a lymphatic endothelial cell.

3. The method according to claim 1, wherein said cellular activity is endothelial cell permeability.

4. The method according to claim 3, wherein said endothelial cell permeability is intercellular or intracellular.

5. The method according to claim 4, wherein said permeability is thrombin-induced vascular endothelial cell permeability.

6. The method according to claim 1, wherein said modulation is up-regulation of protein kinase Cζ activity and said up-regulation is achieved by introducing into said endothelial cell a nucleic acid molecule encoding protein kinase Cζ or functional equivalent, derivative or homologue thereof or the protein kinase Cζ expression product or functional derivative, homologue, analogue, equivalent or mimetic thereof.

7. The method according to claim 1, wherein said modulation is achieved by contacting said endothelial cell with a proteinaceous or non-proteinaceous molecule which modulates transcriptional and/or translational regulation of the protein kinase Cζ gene.

8. The method according to claim 1, wherein said modulation is up-regulation of protein kinase Cζ activity and said up-regulation is achieved by contacting said endothelial cell with a proteinaceous or non-proteinaceous molecule which functions as an agonist of the protein kinase Cζ expression product.

9. The method according to claim 1, wherein said modulation is down-regulation of protein kinase Cζ activity and said down-regulation is achieved by contacting said endothelial cell with a proteinaceous or non-proteinaceous molecule, which functions as an antagonist to the protein kinase Cζ expression product.

10. The method according to claim 9, wherein said molecule is angiopoietin-1 or functional derivative, homologue, analogue, equivalent or mimetic thereof.

11. The method according to claim 9, wherein said molecule is chelerythrine chloride or bisindoylmaleimide I or functional derivative, homologue, analogue, equivalent or mimetic thereof.

12. The method according to claim 9, wherein said molecule is a mutant protein kinase Cζ, which mutant is characterised by substitution of the threonine residue at position 410 of the activation loop to alanine.

13. The method according to claim 1, wherein said endothelial cell activity is modulated in vivo.

14. The method according to claim 13, wherein said endothelial cell activity is modulated in vitro.

15. A method of regulating endothelial cell activity in a mammal, said method comprising modulating the functional activity of protein kinase Cζ in said mammal wherein up-regulating protein kinase Cζ activity to a functionally effective level up-regulates said endothelial cell activity and down-regulating protein kinase Cζ activity to a functionally ineffective level down-regulates said endothelial cell activity.

16. The method according to claim 15, wherein said endothelial cell is a vascular endothelial cell or a lymphatic endothelial cell.

17. The method according to claim 15, wherein said cellular activity is endothelial cell permeability.

18. The method according to claim 17, wherein said endothelial cell permeability is intercellular or intracellular.

19. The method according to claim 18, wherein said permeability is thrombin-induced vascular endothelial cell permeability.

20. The method according to claim 15, wherein said modulation is up-regulation of protein kinase Cζ activity and said up-regulation is achieved by introducing into said endothelial cell a nucleic acid molecule encoding protein kinase Cζ or functional equivalent, derivative or homologue thereof or the protein kinase Cζ expression product or functional derivative, homologue, analogue, equivalent or mimetic thereof.

21. The method according to claim 15, wherein said modulation is achieved by contacting said endothelial cell with a proteinaceous or non-proteinaceous molecule which modulates transcriptional and/or translational regulation of the protein kinase Cζ gene.

22. The method according to claim 15, wherein said modulation is up-regulation of protein kinase Cζ activity and said up-regulation is achieved by contacting said endothelial cell with a proteinaceous or non-proteinaceous molecule which functions as an agonist of the protein kinase Cζ expression product.

23. The method according to claim 15, wherein said modulation is down-regulation of protein kinase Cζ activity and said down-regulation is achieved by contacting said endothelial cell with a proteinaceous or non-proteinaceous molecule which functions as an antagonist to the protein kinase Cζ expression product.

24. The method according to claim 23, wherein said molecule is angiopoietin-1 or functional derivative, homologue, analogue, equivalent or mimetic thereof.

25. The method according to claim 23, wherein said molecule is chelerythrine chloride or bisindoylmaleimide I or functional derivative, homologue, analogue, equivalent or mimetic thereof.

26. The method according to claim 23, wherein said molecule is a mutant protein kinase Cζ which mutant is characterised by substitution of the threonine residue at position 410 of the activation loop to alanine.

27. A method for the treatment and/or prophylaxis of a condition characterised by aberrant, unwanted or otherwise inappropriate endothelial cell activity in a mammal, said method comprising modulating the functional activity of protein kinase Cζ wherein up-regulating protein kinase Cζ activity to a functionally effective level up-regulates said endothelial cell activity and down-regulating protein kinase Cζ activity to a functional ineffective level down-regulates said endothelial cell activity.

28. The method according to claim 25, wherein said endothelial cell is a vascular endothelial cell or lymphatic endothelial cell.

29. The method according to claim 25, wherein said cellular activity is endothelial cell permeability.

30. The method according to claim 29, wherein said endothelial cell permeability is intercellular or intracellular.

31. The method according to claim 30, wherein said permeability is thrombin-induced vascular endothelial cell permeability.

32. The method according to claim 27, wherein said modulation is up-regulation of protein kinase Cζ activity and said up-regulation is achieved by introducing to said mammal a nucleic acid molecule encoding protein kinase Cζ or functional equivalent, derivative or homologue thereof or the protein kinase Cζ expression product or functional derivative, homologue, analogue, equivalent or mimetic thereof.

33. The method according to claim 27, wherein said modulation is achieved by introducing to said mammal a proteinaceous or non-proteinaceous molecule which modulates transcriptional and/or translational regulation of the protein kinase Cζ gene.

34. The method according to claim 27, wherein said modulation is up-regulation of protein kinase Cζ activity and said up-regulation is achieved by introducing to said mammal a proteinaceous or non-proteinaceous molecule which functions as an agonist of the protein kinase Cζ expression product.

35. The method according to claim 27, wherein said modulation is down-regulation of protein kinase Cζ activity and said down-regulation is achieved by introducing to said mammal a proteinaceous or non-proteinaceous molecule which functions as an antagonist to the protein kinase Cζ expression product.

36. The method according to claim 35, wherein said molecule is angiopoietin-1 or functional derivative, homologue, analogue, equivalent or mimetic thereof.

37. The method according to claim 35, wherein said molecule is chelerythrine chloride or bisindoylmaleimide I or functional derivative, homologue, analogue, equivalent or mimetic thereof.

38. The method according to claim 35, wherein said molecule is a mutant protein kinase Cζ, which mutant is characterised by substitution of the threonine residue at position 410 of the activation loop to alanine.

39. The method according to claim 29, wherein said condition is an inflammatory response.

40. The method according to claim 29, wherein said condition is unwanted angiogenesis.

41. The method according to claim 40, wherein said condition is solid tumors, blood born tumors, tumor metastasis, benign tumors, rheumatoid arthritis, Crohn's disease, atherosclerosis, obesity, endometriosis, ocular angiogenic diseases, psoriasis, facial and truncal telangiectasias, or Osler-Webber Rendau syndrome.

42-48. (canceled)
